(19) United States
(12) Patent Application Publication
(10) Pub. No.: US 2011/0178375 A1
(43) Pub. Date: Jul. 21, 2011

(54) REMOTE PHYSIOLOGICAL MONITORING

(75) Inventor: Ian J. FORSTER, Essex (GB)

(73) Assignee: AVERY DENNISON CORPORATION, Pasadena, CA (US)

(21) Appl. No.: 12/689,259

(22) Filed: Jan. 19, 2010

(51) Int. Cl. A61B 5/00

(52) U.S. Cl. 600/301

(57) ABSTRACT

A system for remote physiological monitoring comprises a communication device and a patch. The patch is configured to be removably securable to a body of a biological organism and further configured to monitor a physiological parameter of a biological organism. The communication device is configured to receive communications from the patch. The communication device is further configured to transmit communications to a remote location.

### TECHNICAL FIELD OF THE INVENTION

The disclosed invention relates generally to the field of remote physiological monitoring and more particularly to remote physiological monitoring using a communication device, such as a radio frequency (RF) device to communicate the physiological condition of a subject or patient by measuring various parameters.

### BACKGROUND OF THE INVENTION

Medical professionals may monitor the physiological condition of a patient or subject to determine the health or wellness of the patient or subject. Such physiological monitoring is typically conducted by a medical professional that are directly observing or performing tests on a patient. Such direct observation and testing necessitates that the medical professional and the patient be physically present at the same location. Such a meeting is commonly achieved by either the patient traveling to a doctor's office or other medical facility or by a medical professional traveling to the residence of the patient. With medical professionals and patients both having limited amounts of time, the need to meet at a common location can limit the frequency of opportunities to monitoring the physiological condition of a patient which can often lead to such conditions being ignored. This inactivity can result in a failure of the treatment and cause further set backs to the patient. In addition, prolonged absence of interaction between the health care professional and the patient may further exacerbate a situation leading to the potential exigent need for care and thus increased costs associated with the administering agency and discomfort and risk for the patient.

### BRIEF SUMMARY OF THE INVENTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

A system for remote physiological monitoring comprises a communication device and a patch. The patch is configured to be removably securable to a body of a biological organism such as to the skin or outer surface and further configured to monitor a physiological parameter of a biological organism. The communication device is configured to receive communications from the patch. The communication device is further configured to transmit communications to a remote location.

Another system for remote physiological monitoring comprises a communication device and a patch. The patch is configured to be removably securable to a portion of a body of a biological organism and further configured to cover a wound on a body of a biological organism. The patch includes an observation window to provide visual access to the wound. The communication device is configured to receive an image of the wound and transmit the image to a remote location.

Another system for remote physiological monitoring comprises a communication device and a patch. The patch is configured to be removably securable to a portion of a body of a biological organism and further configured to cover a wound on a body of a biological organism. The patch includes an acoustic pathway to provide acoustic access to the wound. The communication device is configured to direct an acoustic signal to the wound and receive the acoustic signal after the acoustic signal is reflected by the wound. The communication device is further configured to transmit the reflected acoustic signal to a remote location.

In a further exemplary embodiment a method of remotely monitoring a physiological parameter is described and includes the steps of initially providing a RFID apparatus and a communication device to monitor a physiological parameter of a patient. A message is sent from a remote location, such as a health care facility, to the communication device requesting a measurement of a physiological parameter. The requested message is received by the communication device and a signal is transmitted to the RFID apparatus. A measurement of a physiological parameter is initiated by the RFID apparatus and the physiological parameter is measured in response to the requesting message. Then a signal is transmitted containing the measured physiological parameter to the communication device. The signal containing the physiological parameter is sent from the communication to the remote location the signal is received at the remote location.

In the foregoing embodiment once the signal containing the measured parameter is received at the remote location, a second requesting signal can be sent out to potentially reconfirm the measurement contained in the first signal, or the first signal can be used to initiate the sending of a second signal if for example the measurement of the first signal requires further clarification through the use of a second physiological parameter or if other parameters are needed to administer a treatment for the patient.

In a further exemplary embodiment a method of remotely monitoring a physiological parameter is described and includes the steps of initially evaluating a patient's physiological parameter measurements at a remote location, then determining if a treatment is to be applied to the patient and sending a signal to a communication device with instructions to treat the patient. Next, the signal is received at the patient and the signal is transmitted to a RFID assembly. Treatment of the patient is initiated by receiving the signal from the communication device and the on-board treatment device is powered via the RFID to facilitate a treatment and the treatment is delivered to the patient. Finally, delivery of the treatment to the patient is confirmed.

In the foregoing embodiment, the delivery of the treatment can include the release of medicine, providing a stimulus to an implantable device or other suitable treatment necessary for the care of the patient.

Other features and advantages of the present invention will become apparent to those skilled in the art from the following detailed description. It is to be understood, however, that the detailed description of the various embodiments and specific examples, while indicating preferred and other embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

These, as well as other objects and advantages of this invention, will be more completely understood and appreciated by referring to the following more detailed description of the presently preferred exemplary embodiments of the invention in conjunction with the accompanying drawings, of which:

FIG. 1 is a schematic view depicting a system for remote physiological monitoring including a communication device and a patch applied to a forearm of a human;

FIG. 2 is a schematic view depicting a patch for use in a system for remote physiological monitoring, where the patch includes a sensor;

FIG. 3 is a schematic view depicting a patch for use in a system for remote physiological monitoring, where the patch includes a sensor and a treatment device;

FIG. 4 is a schematic view depicting a patch for use in a system for remote physiological monitoring, where the patch includes an optical pathway;

FIG. 5 is a schematic view depicting a patch for use in a system for remote physiological monitoring, where the patch includes an acoustic pathway;

FIG. 6 is a schematic view depicting a patch for use in a system for remote physiological monitoring, where the patch includes a flap covering a passage through the patch;

FIG. 7 is a flowchart illustrating a method of remotely monitoring physiological parameters; and

FIG. 8 is a flowchart illustrating a method of remotely treating a patient.

### DETAILED DESCRIPTION OF THE INVENTION

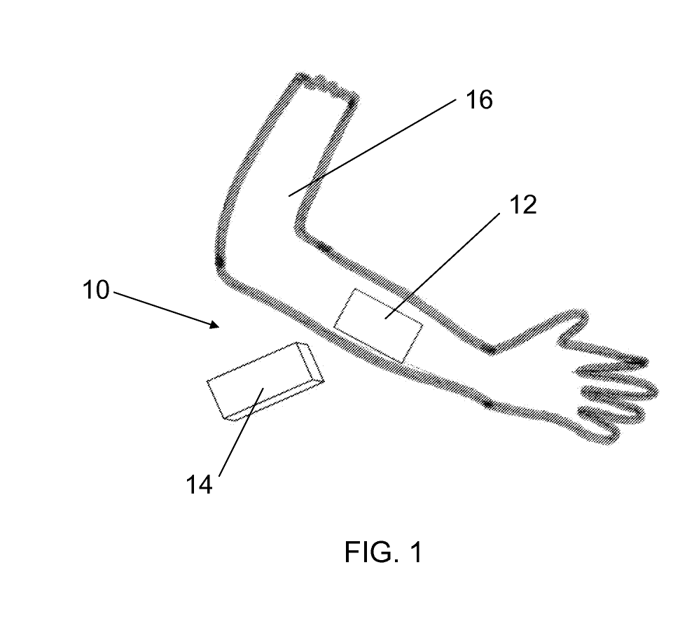

The apparatuses and methods disclosed in this document are described in detail by way of examples and with reference to the figures. Unless otherwise specified, like numbers in figures indicate references to the same, similar, or corresponding elements throughout the figures. It will be appreciated that modifications to disclosed and described examples, arrangements, configurations, components, elements, apparatuses, methods, materials, etc. can be made and may be desired for a specific application. In this disclosure, any identification of specific shapes, materials, techniques, arrangements, etc. are either related to a specific example presented or are merely a general description of such a shape, material, technique, arrangement, etc. Identifications of specific details or examples are not intended to be and should not be construed as mandatory or limiting unless specifically designated as such. Selected examples of apparatuses and methods for remote physiological monitoring of a biological organism such as a human are hereinafter disclosed and described in detail with reference made to FIGS. 1-8.

Physiological monitoring can include the monitoring of at least some of the physiological parameters of a biological organism, such as a human. Physiological parameters include for example heart rate, temperature, perspiration rate, bacteria levels, glucose level, presence of chemical markers, and blood oxygen levels, among others. Physiological monitoring of a human can be useful for a number of purposes. For example, physiological monitoring can assist a doctor or other medical professional in assessing the health and wellness of a patient or diagnosing a disease or condition. By monitoring physiological parameters of interest to a medical professional who is treating the patient, the medical professional can recommend or initiate a course of medical treatment appropriate to address the patient's health, disease or condition.

In another example, physiological monitoring can assist a medical professional in determining if a patient is complying with a prescribed medication regimen. Metabolization of a medication can produce a tell-tale chemical marker that is detectable by monitoring a patient's perspiration or blood. If the tell-tale chemical marker is not detected, this may indicate that the patient is not taking the prescribed medication. If it is determined that the patient is not complying with a prescribed medication regiment, the medical or health care professional can warn the patient of possible dangers and encourage the patient to comply with the prescribed regimen or for the health care professional to suggest other treatment options.

In another example, a caretaker or law enforcement authority can use physiological monitoring to determine whether a person has used an illegal drug or substance. Illegal drug use may be detected by monitoring a person's perspiration, blood, or exhalations. If it is determined that a person has ingested an illegal drug, the caretaker or law enforcement authority can take appropriate actions. Similarly, other illicit or dangerous substances, such as toxins or poisons, can also be detected, such as to alert one to a possible suicide situation.

Monitoring a human's physiological parameters from a remote location can increase the frequency, efficiency, and usefulness of such monitoring. For example, a medical professional can more frequently monitor a patient's health or adherence to a medication or treatment regimen if such monitoring can be done remotely. Remote monitoring reduces the need for the doctor or health care professional and the patient to meet at a common location.

In another example, if a child or elderly person suffers from volatile glucose levels, a caregiver commonly monitors the child's or elderly person's glucose level to insure that the levels are within acceptable range. If the caregiver can monitor the glucose level from a remote location, it reduces the need for the caregiver to be located in the same location as the child or elderly person.

In one example, a system for remotely monitoring a physiological parameter of a human includes positioning a sensor on or near the body of the human to sense or read a physiological parameter. A communication device can be arranged to receive communications from the sensor, where the communications can include information on the monitored physiological parameter. The communication device can further be configured to receive communications from and transmit communications to remote locations to facilitate the transmission of information regarding the physiological parameter to the remote location.

Such a remote location can be a doctor's office, hospital or other such health facility, a law enforcement office, a computer or server designated as a central location for storing physiological measurements of groups of people such as patients or probationers, and the like.

It should be understood that a remote location includes any location where the person interested in a subject's physiological parameter measurement does not have direct access to the subject. For example, for a patient admitted to a hospital, the remote location could be a computer or server located in the patient's room that is arranged to receive and store physiological measurements of the patient. Likewise a remote location could be a central monitoring location while the patient or subject remains at home or is circulating freely outside of a particular location.

As schematically illustrated in FIG. 1, one example of a remote physiological monitoring system 10 can include a patch 12 and a communication device 14. The patch 12 can be configured to be removably secured to a person's body 16. For example, the patch 12 can be adhered to the person's forearm as shown. In another example, the patch 12 can be positioned near, but not directly on, a person's body so as to facilitate physiological monitoring of the person. For example, the patch 12 can be secured to a person's clothing proximate to the person's chest such that the patch 12 can monitor the person's heart rate. As will be further described, the patch 12 can include a monitoring device configured to monitor a specific physiological parameter of the person. For example, the patch 12 can be configured to monitor a person's heart rate, temperature, perspiration rate, glucose level, blood oxygen levels, or other such parameters. The patch 12 can be further configured to communicate information regarding the monitored physiological parameter to the communication device 14. The communication device 14 in turn can be configured to receive or otherwise capture the information communicated by the patch 12 regarding the monitored physiological parameter.

In one example, the patch 12 is configured to communicate with the communication device 14 through wireless communication technology, and the communication device 14 is configured to communicate with the patch through such wireless communication technology. Examples of wireless communication technologies include, but are not limited to, near field communication; short-range radio frequency communication system, such as those that operate on standards such as the IEEE 802 family of wireless communication protocols, including for example, but not limited to 802.11 and 802.15; and infrared signal communication, among others.

In one example, the communication device 14 can be a multimedia mobile phone or personal digital assistant (PDA) equipped with near field communication capabilities. Although the communication device 14 is described as a mobile phone or personal digital assistant, it will be understood that the communication device 14 can be any device capable of receiving and transmitting information or data. For example, the communication device 14 can be a device that is customized and designed specifically to function as a communication device 14 in a remote physiological monitoring system 10. The patch 12 can include a radio frequency identification (RFID) apparatus 18 to facilitate communication. In such an example, the communication device 14 and patch 12 can communicate by placing the communication device 14 in close proximity to the patch 12, as shown in FIG. 1.

In one example, the communication device 14 can initiate communication with the RFID apparatus 18 by sending a signal to the RFID apparatus 18 that results in the RFID apparatus 18 responding with information or data regarding the monitored physiological parameter. In another example, the RFID apparatus 18 can sense that the communication device 14 is positioned proximate to the RFID apparatus 18 and initiate a signal to the communication device 14 with information or data regarding the monitored physiological parameter.

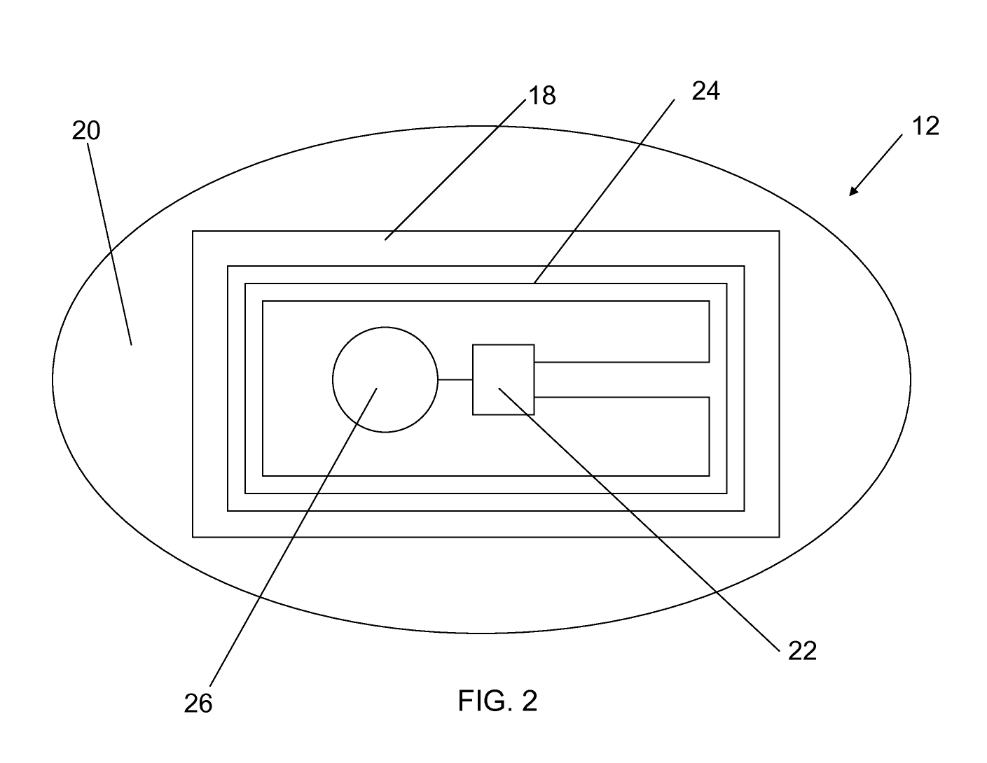

As schematically illustrated in FIG. 2, the patch 12 can include an adhesive portion 20 for removably securing the patch 12 to the body 16 of a person. The RFID apparatus 18 can include a RFID chip 22 and an antenna 24. The patch 12 can further include a sensor 26 that is in electrical communication with the patch 12 to facilitate monitoring of a physiological parameter. The RFID chip 22 can be a high-frequency RFID chip 22 with a unique identifier to facilitate accurate communication with the communication device 14. The antenna 24 is in electrical communication with the RFID chip 22 and is configured to receive signals from devices such as the communications device 14 and configured to transmit signals handled by the RFID chip 22.

The sensor 26 can be configured in a number of ways depending on the physiological parameter to be monitored. For example, if a glucose level is to be monitored, the sensor 26 can include an infrared radiation source used to inspect the skin of a person and determine a glucose level. In another example, if blood oxygen level is to be monitored, the sensor 26 can include a light emitting diode and a photodiode arranged to measure a person's blood oxygen level.

The sensor may also measure parameters associated with a wound or condition of a person's body. For example, the sensor may emit and receive and acoustic signal; it may measure the electrical conductivity between two or more points at a range of frequencies and at DC, and the measurements can be relayed back to the communication device via the RFID chip. Alternatively, the RFID chip may emit a signal, such as an acoustic response, into the wound area, and the phone may use its own sensors, such as its microphone, to determine the response.

Physiological parameters measured by the physiological monitoring system 10 can be transmitted to medical professionals, caretakers, and law enforcement officers that are located remotely from the person. In one example, the sensor 26 can be configured to measure the body temperature of a patient. Such a temperature measurement may be taken by positioning the sensor 26 in direct contact with the body 16 of a person. It will be understood that the sensor 26 can be placed in direct contact with the person by securing the patch 12 to a person's body 16 using the adhesive portion 20 if the patch 12.

At any desired time, the person wearing the patch 12 can initiate a physiological parameter measurement by moving the communication device 14 into close proximity of the patch 12 as shown in FIG. 1. Such proximity can direct the sensor 26 to read or measure the person's body temperature. Once the temperature is measured, the sensor 26 can communicate the measurement to the RFID chip 22 through the electrical connection between the sensor 26 and the RFID chip 22. The RFID chip 22 can then communicate the measurement to the communication device 14 by using the antenna 24 to transmit a signal containing the measurement to the communication device 14. In such an arrangement, the communication device 14 can function as an RFID reader. Once the measurement is received by the communication device 14, the measurement can be further transmitted to a remote location for immediate viewing by a medical professional or stored for future viewing by a medical professional providing flexibility for the medical professional.

When the communication device 14 is a mobile phone or a personal digital assistant, the measurement can be transmitted to the remote location in a number of forms. For example, the measurement can be transmitted as raw data, as a text message, as an electronic mail message, or similar method of transmission. While the measurements can be transmitted using wireless technologies, the measurements can also be transmitted by other communication methods. For example, the communication device 14 can be linked to a computer that transmits the measurement over an intranet or the Internet.

A physiological parameter measurement can be initiated remotely by a medical professional. For example, a medical professional that would like a physiological parameter measurement from a patient can contact the patient and request a measurement. Such contact can be achieved by the medical professional initiating a call, sending an electronic mail, or sending a text message to the patient requesting the patient perform a physiological parameter measurement. Upon receiving the request, the patient can move the communication device 14 into proximity of the patch 12 to initiate the measurement. When the communication device 14 is a mobile phone or a personal digital assistant, the patient can receive the request through the mobile phone or personal digital assistant and immediately initiate a measurement to be transmitted back to the medical professional.

In another example, a physiological parameter measurement can be initiated remotely by a medical professional sending a signal to the communication device 14 instructing the communication device 14 to automatically attempt a physiological parameter measurement. The communication device 14 responds by sending a signal to the RFID apparatus 18 to initiate the measurement. Any successful measurement can be transmitted to the communication device 14 and further communicated to the medical professional. In such an example, the patient need not take any proactive actions and may not even be aware that a measurement is taken and communicated to the medical professional.

Although this disclosure describes medical professionals or patients initiating the measurement of physiological parameters, it will be understood that such measurements can be automated. For example, the communication device 14 can be programmed to initiate measurements at set times or intervals. In another example, the RFID apparatus 18 can be programmed to initiate measurements at set times or intervals. In yet another example, a remote computer or other such remote device can be programmed to send signals to the communication device 14 to initiate measurements at set times or intervals.

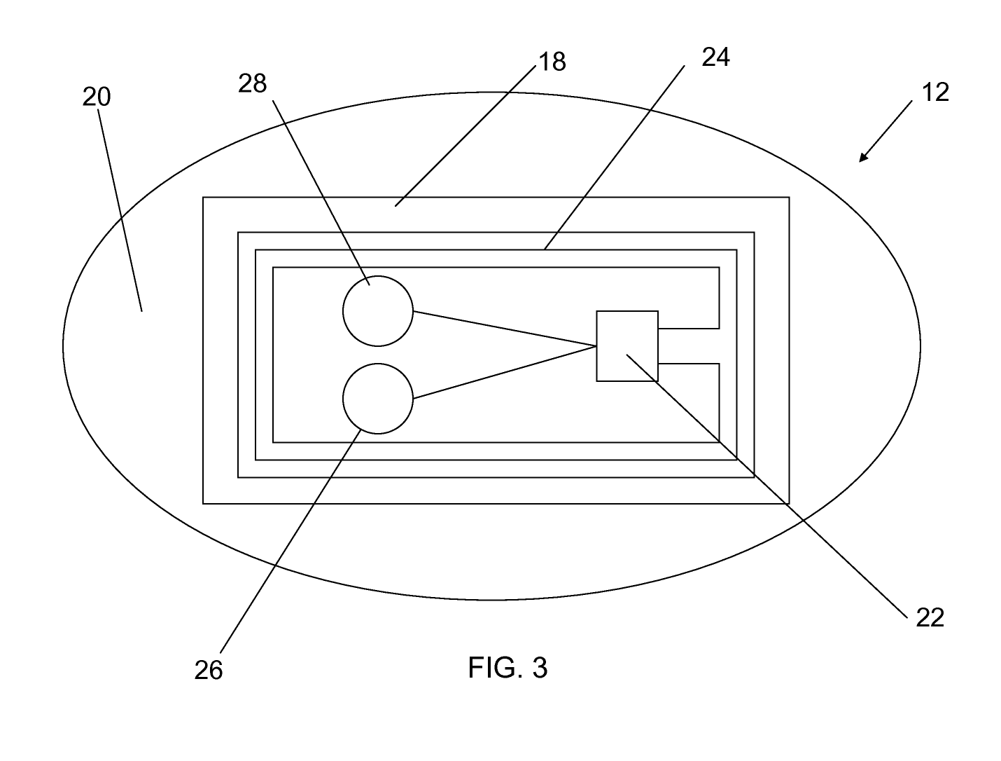

In addition to monitoring physiological parameters, a patch 12 can be configured to selectively administer treatment to a patient. For example, the patch 12 can be a dressing or bandage configured to cover a cut, burn, or other such wound on the surface of a person's body 16. As schematically illustrated in FIG. 3, in addition to the sensor 26, the patch 12 can include a treatment device 28 in electrical communication with the RFID chip 22. The patch 12 can be arranged to adhere to a person's body 16 so that the patch 12 covers a wound and the sensor 26 and treatment device 28 are positioned above and proximate to the wound. The sensor 26 can be configured to detect or measure the level of bacteria in the wound. The level of bacteria in a wound can be an indicator of how well the wound is healing and whether the wound is infected.

The treatment device 28 can be configured to apply an anti-bacterial treatment to the wound. In one example, the treatment device 28 can include an ultraviolet or blue light source. When such a light source is illuminated and directed to the wound, the light can administer an anti-bacterial treatment that lowers the level of bacteria in the wound. In another example, the treatment device 28 can include an ozone-generating component. When ozone is generated and comes into contact with the wound, the level of bacteria in the wound can be lowered. In another example, the treatment device 28 can be a device that controllably releases an anti-bacterial agent or ointment onto the wound upon actuation of the device. In such an example, the treatment device 28 can include a reservoir to retain anti-bacterial ointment and a piezo electrical device to control the release of the anti-bacterial agent upon actuation of the piezo electrical device.

The sensor 26 can be directed to measure the level of bacteria in the wound and communicate the measurement to a medical professional located at a remote location. Upon reviewing the bacterial level measurement, the medical professional can evaluate the level of bacteria in the wound and optionally recommend treating the wound with an anti-bacterial agent. The measuring of the bacterial level in the wound can be initiated by the patient by bringing the communication device 14 into proximity of the patch 12. A signal sent to the RFID apparatus 18 by the communication device 14 can cause the RFID apparatus 18 to direct the sensor 26 to measure the bacterial level. The measurement can be transmitted by the RFID apparatus 18 to the communication device 14, which further transmits the measurement to the remote medical professional. If appropriate, the medical professional can send a message to the communication device 14 to initiate an anti-bacterial treatment of the wound.

In one example, the communication device 14 is configured to receive a message from the medical professional and automatically sending a signal to the patch 12 to administer an anti-bacterial treatment. In such an example, the communication device 14 sends a signal to the RFID chip 22 with instructions to administer an anti-bacterial treatment. The RFID chip 22 directs electrical power to the treatment device 28, which in turn directs an anti-bacterial agent to the wound. When the treatment device 28 is an ultraviolet or blue light source, the electrical power is directed to illuminating the light source. When the treatment device 28 is an ozone-generating component, the electrical power is directed to generating ozone. When the treatment device 28 is configured to release an anti-bacterial ointment, the electrical power is directed to actuate the piezo electrical device and release the ointment.

In one example, the patch 12 can include a power source, such as a battery, from which electrical power can be directed to the treatment device 28 by the RFID apparatus 18. In another example, a portion of the radio signal transmitted from the communication device 14 to the RFID apparatus 18 can be transformed to electrical power by the RFID apparatus 18 and directed to the treatment device 28. Thus, it will be understood that in this and other examples the RFID apparatus 18 can be an active, a semi-passive, or a passive RFID apparatus 18.

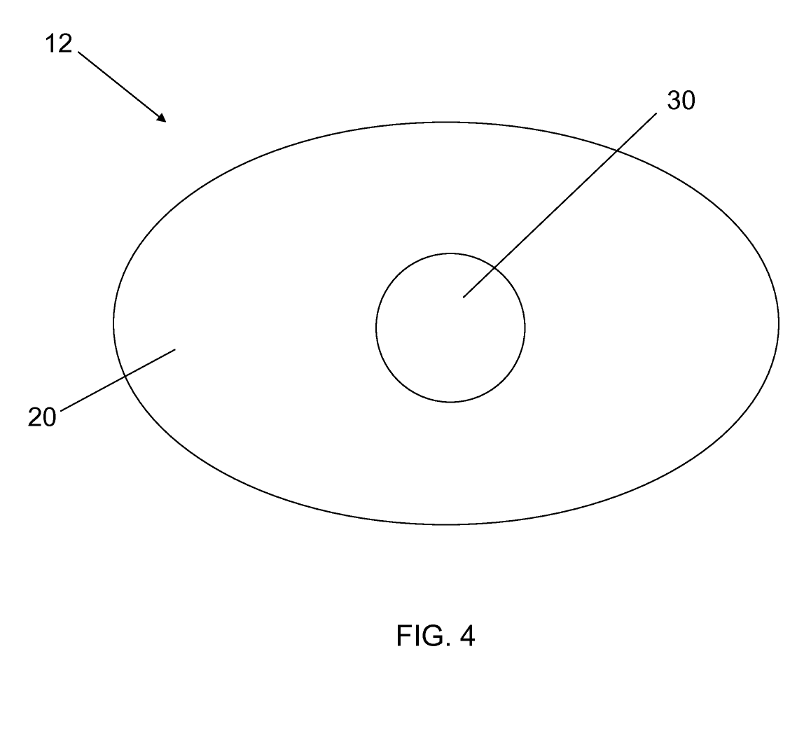

The patch 12 can be configured to allow for direct observation or inspection of a wound to determine the condition of the wound or how well the wound is healing. In one example, as schematically illustrated in FIG. 4, the patch 12 includes an observation window 30 for visual inspection of the wound. The patch 12 can be secured to the body 16 of a patient so that a wound is covered and the observation window 30 is positioned over the wound. The observation window 30 can be a fresnel lens, a holographic lens, or any other such component that provides a pathway for visual inspection of the wound. In addition, the pathway or observation window may only allow an inspection of the wound at specific wavelengths, and the wavelengths may be outside the normal visual range for a person but inside the sensing range of a camera or other sensor system inside the communication device. For example, the window may transmit infra-red wavelengths. In this way the window allows access to the wound status to a suitably equipped medical professional but does not display the wound to either the patient or others who may find the image distressing. Alternatively, the window may be a form of shutter, such as a liquid crystal cell, which only allows a visual pathway to be established when the communication device sends a signal to a control circuit, such as an RFID device, embedded in the dressing. Illumination for the image may be provided from a suitable light source, such as a Light Emitting Diode (LED), integrated into the dressing, with the emission of the light controlled by the communication device. In an alternate embodiment, the power for the optical source is rectified from the RF emissions of the communication device, such as the transmission used to establish long range communication with a host system or the transmission used for short range communication such as that used to read an RFID device.

Alternately, the window and the display can be used to provide a visual cue to the patient, such as by changing color or displaying a basic message alerting the patient to seek assistance or clarification, or that the device itself is not functioning properly. The display can be created through the use of electrophoretic particles, electrochomic films or other suitable means to create a display, color or visual cue.

When the communication device 14 is a mobile phone, personal digital assistant, or other device capable of capturing a photograph, the patient can use the communication device 14 to capture a photograph of the wound through the observation window 30. Once captured, the patient can use the communication device 14 to transmit the photograph to a remote medical professional for evaluation of the healing process of the wound. When the observation window 30 is a lens, the lens can be configured to allow for magnification of the wound surface so that the medical professional can evaluate a high-quality photograph. Upon inspection of the photograph, the medical professional can determine if any additional medical treatment is necessary. For example, the medical professional can require an anti-bacterial treatment or require the patient make an appointment with the medical professional to further inspect or treat the wound.

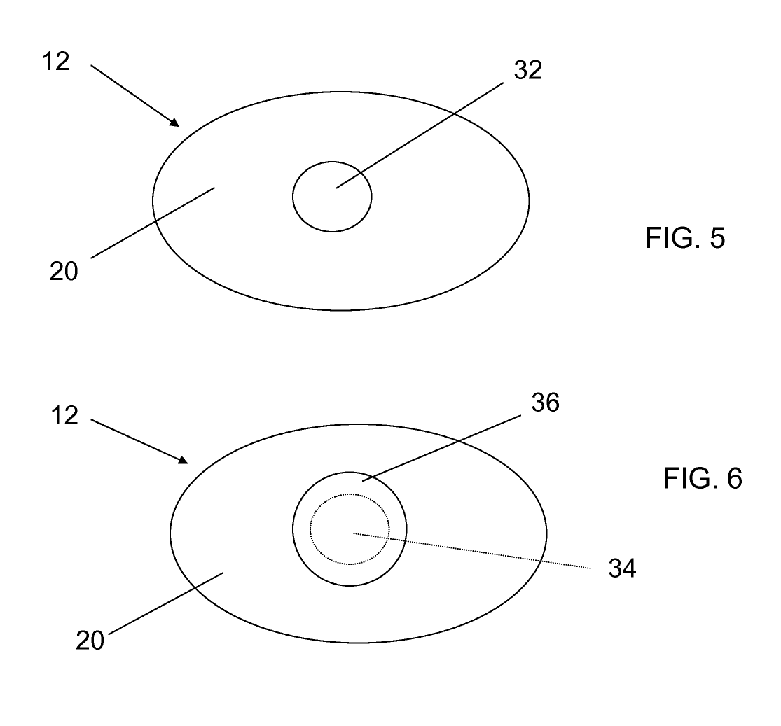

In another example, as schematically illustrated in FIG. 5, the patch 12 can include an acoustic pathway 32 positioned so that when the patch 12 is covering a wound, the wound can be inspected by acoustic signals through the acoustic pathway 32. When the communication device 14 is a mobile phone, personal digital assistant, or other device capable of emitting and receiving acoustic signals, the patient can use the communication device 14 to inspect the wound with acoustic signals. In one example, a mobile phone includes both a speaker for emitting acoustic signals and a microphone for receiving acoustic signals. Therefore, a mobile phone can be utilized to emit an initial acoustic signal through the acoustic pathway 32 directed at the wound and receive a return acoustic signal once the initial acoustic signal has reflected off the wound. The change in the return signal as compared to the initial acoustic signal can be analyzed to determine the tautness or tension of the surface of the wound. Such analysis can be used to evaluate the healing process of the wound. It will be understood that the acoustic pathway 32 can be made of any material or medium that allows for the propagation of acoustic signals.

In one example, schematically illustrated in FIG. 6, the patch 12 can comprise an open passage 34 through the patch 12 and a flap 36 configured to selectively cover the open passage 34. The flap 36 can be selectively removed to expose the open passage 34. Once the open passage 34 is exposed, the wound can be inspected by acoustic signals as previously described.

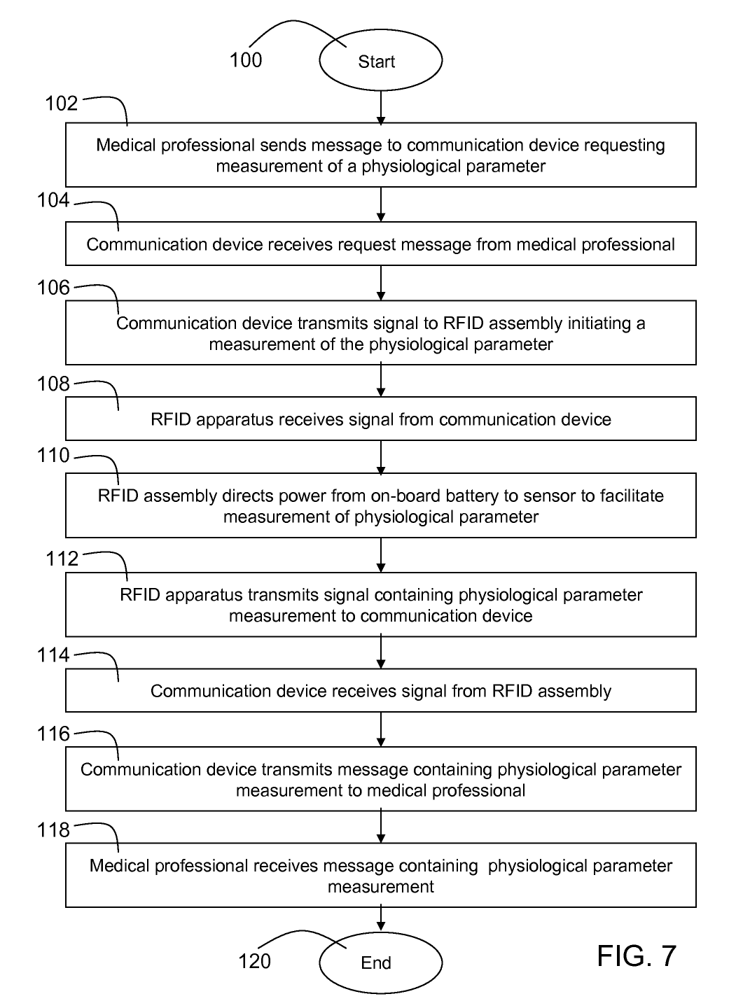

In one example method of remotely monitoring a physiological parameter, a medical professional can remotely request a measurement of a physiological parameter and can further receive the result of the measurement of the physiological parameter. With reference to FIG. 7, such a method begins at start block 100. At process block 102, a medical professional sends a message to the communication device requesting a measurement of a physiological parameter. At process block 104, the communication device receives the request message from the medical professional. At process block 106, the communication device transmits a signal to the RFID apparatus initiating a measurement of the physiological parameter. At process block 108, the RFID apparatus receives the signal from the communication device. At process block 110, the RFID apparatus directs power from an on-board battery to the sensor to facilitate measurement of the physiological parameter. At process block 112, the RFID transmits a signal containing the physiological parameter measurement to the communication device. At process block 114, the communication device receives the signal from the RFID assembly. At process block 116, the communication device transmits a message containing the physiological parameter measurement to the medical professional. At process block 118, the medical professional receives the message containing the physiological measurement. Execution of the method ends at end block 120 once the message is confirmed and the information received is sufficient or satisfactory to terminate the process. Alternatively, a second requesting message can be sent from the remote location where the medical professional resides based on the first signal that is received to either confirm the first measurement that has been received in the signal or to take a second, different measurement in order to further clarify the condition of the patient and to potentially order additional treatment steps. A display of the condition of the patient or measurement taken can be rendered at the location of the measurement to also indicate to the patient the results of the measurement, such as visual cues or possibly audible cues.

The foregoing method can also generate signals at the local site to create a display that may have one or more visual or audible cues for the patient, such as a simple message composed of alpha and or numeric characters, colors, symbols or the like such that the patient is aware of the results or if the device needs corrective action such as it is not receiving signals or otherwise needs to be adjusted.

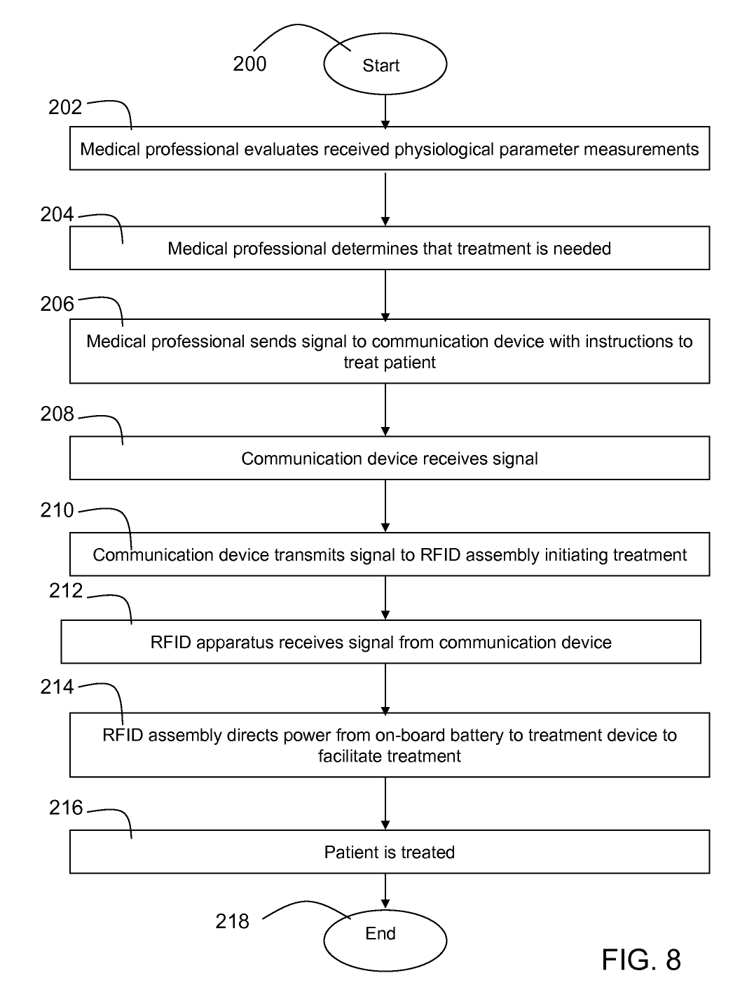

In an example method of remotely treating a patient, a medical professional can remotely initiate treatment of a patient. With reference to FIG. 8, such a method starts at start block 200. At process block 202, a medical professional evaluating a patient's physiological parameter measurements. At process block 204, the medical professional determines that a treatment is to be applied to the patient. At process block 206, the medical professional sends a signal to the communication device with instructions to treat the patient. At process block 208, the communication device receives the signal. At process block 210, the communication device transmits a signal to the RFID assembly initiating treatment of the patient. At process block 212, the RFID assembly receives the signal from the communication device. At process block 214, the RFID assembly directs power from the on-board battery to the treatment device to facilitate treatment. At process block 216, the patient is treated. Execution of the method ends at end block 218. The treatment device can include release of medicines; provide stimulus to implantable devices or such other treatment as may be enabled by the devices of the present system in order to provide the necessary care for the patient.

While the foregoing methods have been described with an on-board battery system, it should be understood that the system may receive power without a battery such as through the use of a power or multiple antenna configuration, capacitor or other means known to provide suitable power to the device.

The foregoing description of examples has been presented for purposes of illustration and description. It is not intended to be exhaustive or limiting to the forms described. Numerous modifications are possible in light of the above teachings. Some of those modifications have been discussed, and others will be understood by those skilled in the art. The examples were chosen and described in order to best illustrate principles of various examples as are suited to particular uses contemplated. The scope is, of course, not limited to the examples set forth herein, but can be employed in any number of applications and equivalent devices by those of ordinary skill in the art.

What is claimed is:

1. A system for remote physiological monitoring comprising:
a communication device and a patch; the patch is configured to be removably securable to a biological organism and is further configured to monitor a physiological parameter of the biological organism;
the communication device is configured to receive communications from the patch; and
the communication device is further configured to transmit communications to a remote location.

2. The system as recited in claim 1, wherein the physiological parameter is selected from a group including heart rate, temperature, perspiration rate, bacteria levels, glucose level, presence of chemical markers, blood oxygen levels, and combinations thereof.

3. The system as recited in claim 1, wherein a visual or audible cue is displayed by the communication device.

4. The system as recited in claim 1, wherein communications are generated on an intermittent basis in response to timed signals.

5. The system as recited in claim 1, wherein the system includes a sensor to monitor at least one of the physiological parameters and the sensor generates at least one of a audible or visual message relating to the physiological parameter.

6. The system as recited in claim 1, wherein the patch includes an observation window.

7. The system as recited in claim 6, wherein the observation window provides an image at a predetermined wave length.

8. A system for remote physiological monitoring, comprising:
a communication device and a patch;
the patch is configured to be removably securable to a portion of a body of a biological organism and further configured to cover a wound on a body of a biological organism;
the patch includes an observation window to provide visual access to the wound; and
the communication device is configured to receive an image of the wound and transmit the image to a remote location.

9. The system as recited in claim 8, wherein the observation window permits viewing in a predetermined wavelength.

10. The system as recited in claim 9, wherein the wavelength is infrared.

11. The system as recited in claim 8, wherein the patch includes an RFID apparatus for communication with the communication device.

12. A system for remote physiological monitoring, comprising:
a communication device and a patch;
the patch is configured to be removably securable to a portion of a body of a biological organism and is further configured to cover a wound on a body of a biological organism;
the patch includes an acoustic pathway to provide acoustic access to the wound;
the communication device is configured to direct an acoustic signal to the wound and receive the acoustic signal after the acoustic signal is reflected by the wound to create a reflected acoustic signal; and
the communication device is further configured to transmit the reflected acoustic signal to a remote location.

13. A method of remotely monitoring a physiological parameter, the method including the steps of:
providing a RFID apparatus and a communication device to monitor a physiological parameter of a patient;
sending a message from a remote location to the communication device requesting a measurement of a physiological parameter;
receiving the requesting message by the communication device;
transmitting a signal from the communication device to the RFID apparatus;
initiating a measurement of a physiological parameter based on the transmitted signal by the RFID apparatus;
measuring the physiological parameter in response to the requesting message;
transmitting a signal containing the physiological parameter to the communication device;
sending the signal containing the physiological parameter from the communication to the remote location; and
receiving the signal at the remote location.

14. The method as recited in claim 13, including a further step of confirming the signal at the remote location after the step of receiving the signal and sending a second requesting message seeking confirmation of the measurement.

15. The method as recited in claim 13, including a further step of sending a second requesting messages after the step of receiving the signal at the remote location seeking a measurement on a distinct physiological parameter based on the receiving the first signal.

16. The method as recited in claim 13, wherein the RFID apparatus provides a visual or audible signal relating to results of the measurement of the physiological parameter.

17. The method as recited in claim 13, wherein the RFID apparatus provides a visual or audible signal relating to the operative status of the RFID apparatus.

18. The method as recited in claim 13, wherein the signals are transmitted in regular intervals.

19. The method as recited in claim 13, wherein the physiological parameter is selected from a group including heart rate, temperature, perspiration rate, bacteria levels, glucose level, presence of chemical markers, blood oxygen levels, and combinations thereof.

20. A method of remotely monitoring a physiological parameter, comprising the steps of:
evaluating a patient's physiological parameter measurements at a remote location;
determining a treatment is to be applied to the patient based on the step of evaluating;
sending a signal from the remote location to a communication device with instructions to treat the patient;
receiving the signal at the communication device;
transmitting a signal from the communication device to a RFID apparatus;
initiating treatment of the patient by receiving the signal from the communication device at the RFID apparatus;
powering an on-board treatment device via the RFID apparatus to facilitate a treatment;
delivering treatment to the patient; and
confirming delivery of the treatment to the patient by transmitting a signal from the RFID apparatus to the communication device and the remote location.

21. The method as recited in claim 20, including a further step of transmitting a second signal to the RFID assembly after the step of confirming delivery of the treatment to the patient.

22. The method as recited in claim 21, wherein the second signal includes information about providing a second treatment to the patient.

23. The method as recited in claim 21, wherein the treatment is selected from a group including medicines, stimulus for implantable devices or combinations thereof.

24. The method as recited in claim 20, wherein the delivery of treatments is provided in regular time increments.
